(19) United States
(12) Patent Application Publication
(10) Pub. No.: US 2013/0030285 A1
(43) Pub. Date: Jan. 31, 2013

(54) SYSTEM AND METHOD FOR INTEGRATING MULTIPLE DATA SOURCES INTO AN X-RAY IMAGE REFERENTIAL

(71) Applicants: Régis Yves Vaillant, Villebon sur Yvette (FR); Adrian Francis Warner, Delafield, WI (US); Claudio Patricio Mejia, Chicago, IL (US); Ion Petros Pappas, Budapest (HU)

(72) Inventors: Régis Yves Vaillant, Villebon sur Yvette (FR); Adrian Francis Warner, Delafield, WI (US); Claudio Patricio Mejia, Chicago, IL (US); Ion Petros Pappas, Budapest (HU)

(73) Assignee: General Electric Company, Schenectady, NY (US)

(21) Appl. No.: 13/191,313

(22) Filed: Jul. 26, 2011

(51) Int. Cl. A61B 5/05; A61B 6/00

(52) U.S. Cl. 600/424; 600/427

(57) ABSTRACT

A method for integrating multiple data sources into an X-ray image referential includes generating an X-ray image of a subject. The method also includes collecting information from sensors of a catheter disposed within the subject. The method further includes generating the X-ray image referential on a coordinate system by merging a model of an anatomy and the collected information from the sensors of the catheter to the X-ray image, wherein the X-ray image referential includes a display of a trace of a catheter. In addition, the method includes displaying the X-ray image referential on a display.

### BACKGROUND OF THE INVENTION

The subject matter disclosed herein relates to X-ray imaging systems and more particularly to a method and system for integrating multiple data sources with X-ray images generated by the X-ray imaging systems.

X-ray imaging is a tool widely used in the diagnosis and treatment (e.g., electrophysiological treatments) of heart pathologies. For example, in certain interventional procedures or operations related to improving the electrical conduction within a heart (e.g., ablation of auricular fibrillation), the practitioner may employ catheters and/or guides within cavities of the heart. These techniques are employed to avoid the need for heavy surgical interventions. X-ray imaging (e.g., fluoroscopy) helps monitor the progress of the catheter as the practitioner guides the catheter within the patient. However, the X-ray imaging may not provide all of the necessary information (e.g., electrical field within the cardiac chambers, parameters related to the catheter, 3D image information related to the heart and surrounding anatomical structure) for the intervention. Attempts to integrate all of the necessary information into a useful information model have added cost to the procedure. In addition, these information models are difficult and time consuming to navigate, especially if the information model includes switching between multiple data sources.

There is a need, therefore, for providing all of the information relative to an interventional procedure in a user-friendly manner. There is a particular need for a technique that can collect the relevant information from multiple sources into a single dataset that provides easy access, display, and editing of the information for the practitioner to look at prior to, during, or after the procedure.

### BRIEF DESCRIPTION OF THE INVENTION

In accordance with certain aspects of the present techniques, a method for integrating multiple data sources into an X-ray image referential includes generating an X-ray image of a subject. The method also includes collecting information from sensors of a catheter disposed within the subject. The method further includes generating the X-ray image referential on a coordinate system by merging a model of an anatomy and the collected information from the sensors of the catheter to the X-ray image, wherein the X-ray image referential includes a display of a trace of a catheter. In addition, the method includes displaying the X-ray image referential on a display.

In accordance other aspects of the techniques, a system includes an imaging device configured to generate an X-ray image of a subject. The system also includes a catheter disposed within the subject, wherein the catheter includes sensors configured to collect information. The system further includes a processor configured to generate an X-ray image referential on an organ coordinate system by merging a model of an anatomy and collected information from the sensors of the catheter to the X-ray image, wherein the X-ray image referential includes a display of a trace of the catheter. In addition, the system includes a display configured to display the X-ray image referential.

Moreover, in accordance with other aspects of the techniques, a computer program, stored on a tangible, non-transitory computer readable medium, for integrating multiple data sources into an X-ray image referential is constructed and arranged to receive an X-ray image of a subject and collected information from sensors of a catheter disposed within the subject. The program is also constructed and arranged to generate the X-ray image referential on an organ coordinate system by merging a model of an anatomy and the collected information from the sensors of the catheter to the X-ray image, wherein the X-ray image referential includes a display of a trace of the catheter. The program is also constructed and arranged to display the X-ray image referential.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic view of an exemplary system for collecting data from multiple sources (e.g., imaging system with one source and one detector) and integrating the data into a single dataset, equipped in accordance with aspects of the present disclosure;

FIG. 2 is a schematic view of an exemplary system for collecting data from multiple sources (e.g., imaging system with two sources and two detectors) and integrating the data into a single dataset, in accordance with aspects of the present disclosure;

FIG. 3 is a diagrammatical view of an exemplary system for collecting and integrating data, in accordance with aspects of the present disclosure;

FIG. 4 is a schematic view of the exemplary system for collecting and integrating data, equipped in accordance with aspects of the present disclosure;

FIG. 5 is an example of a screen for an X-ray image referential;

FIG. 6 is an example of a table from the X-ray image referential;

FIG. 7 is a flow diagram of an exemplary method for integrating multiple data sources into an X-ray image referential, in accordance with aspects of the present disclosure; and

FIG. 8 is a process diagram of an exemplary program for integrating multiple data sources into an X-ray image referential, in accordance with aspects of the present disclosure.

### DETAILED DESCRIPTION OF THE INVENTION

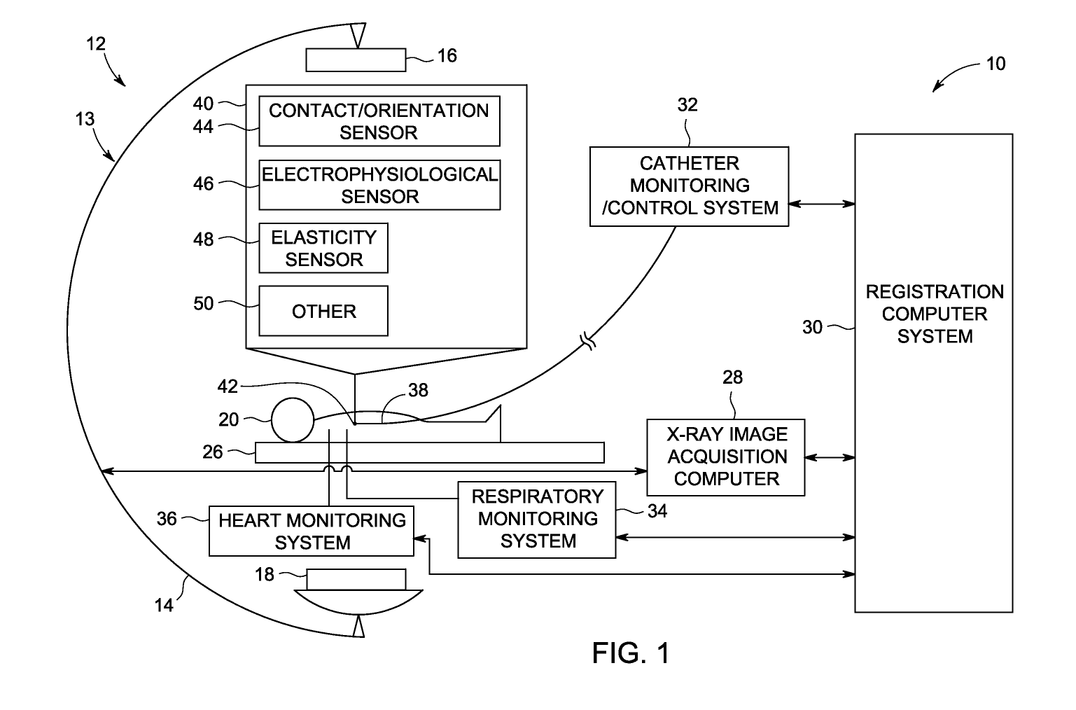

Referring now to FIG. 1, an exemplary system 10 for collecting data from multiple data sources and integrating the data into a single dataset is illustrated. In particular, as described in greater detail below, an X-ray image may be generated and information collected from multiple sources (e.g., catheter, respiratory monitoring system, heart monitoring system, etc.). The collected information and a model of an anatomy may be merged with the generated X-ray image to generate an X-ray image information model or X-ray image referential that collects the data from the multiple sources into a single dataset on a coordinate system (e.g., organ coordinate system). The X-ray image referential serves as a single reference or access point for the generated image and any collected information related to the image. For example, the X-ray image referential may include the X-ray image, 3D and/or 4D information related to the image, a trace of a catheter within the body of a subject (e.g., in a heart), a current and past position and orientation of the catheter relative to the X-ray image, electrophysiological data at certain points along the trace, parameters related to the catheter at certain points along the trace, and other information all within the X-ray image referential. The X-ray image referential may also include a hierarchical structure that allows immediate visualization or direct access to the collected information (e.g., via menus, tables, links, etc.) associated with each point along the trace of the catheter.

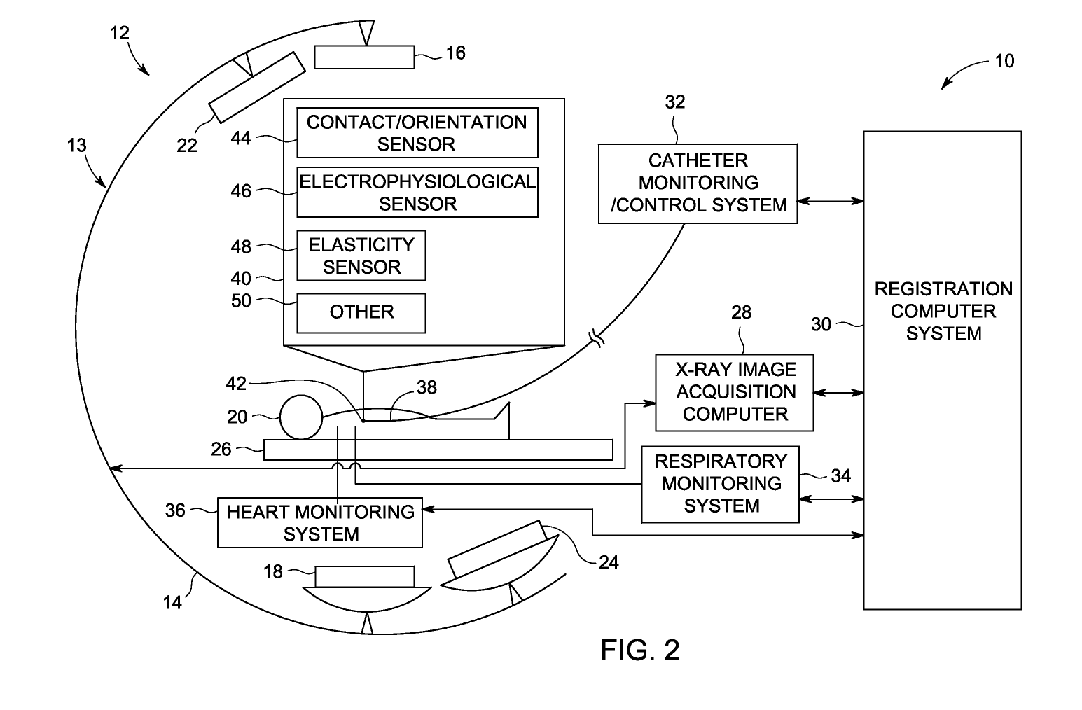

In the illustrated embodiment, the system 10 includes an X-ray imaging system 12 (e.g., X-ray fluoroscopy system) for processing and acquiring image data. The X-ray imaging system 12 includes an imaging device 13 illustrated as a C-arm system that includes a C-arm 14, an X-ray radiation source 16, and an X-ray detector 18. The X-ray radiation source 16 is mounted on the C-arm 14, and the X-ray detector 18 is mounted on the C-arm 14 in an opposing location from the X-ray radiation source 16. While in some systems the X-ray radiation source 16 and the X-ray detector 18 may be fixed, in a typical fluoroscopy system the C-arm 14 allows for movement of the X-ray radiation source 16 and the X-ray detector 18 about a patient or subject 20. Alternatively, as illustrated in FIG. 2, the X-ray imaging device 13 may include an additional X-ray radiation source 22 mounted on the C-arm 14 in an opposing location from an additional X-ray detector 24 also mounted on the C-arm 14 to enable the generation of X-ray images from different angles. Due to the multiple X-ray radiation sources 16 and 22 and X-ray detectors 18 and 24, the X-ray imaging device 13 in FIG. 2 may enable bi-plane imaging to generate X-ray images at different angles which may be used in the techniques discussed below. Alternatively, multiple X-ray imaging devices 13 may be used for bi-plane imaging. In operation, the X-ray radiation source 16 or 22 emits a stream of radiation suitable for X-ray fluoroscopy. The X-ray detector 18 or 24 receives a portion of the stream of radiation from the X-ray source 16 or 22 that passes through the subject 20 positioned on a table 26. The X-ray detector 18 or 24 produces electrical signals that represent the intensity of the radiation stream. As those of ordinary skill in the art will appreciate, these signals are suitably acquired and processed to reconstruct an image of features within the subject 20. In particular, the X-ray imaging system 12 also includes an X-ray image acquisition computer 28 that acquires the data collected from the X-ray detector 18 or 24. In certain embodiments, the X-ray image acquisition computer 28 may include a controller configured to control the operation of the X-ray imaging device 13. In particular, the X-ray image acquisition computer 28 includes processing circuitry to generate 2-D images of an anatomical region (e.g., the heart and surrounding area) utilizing the data from the X-ray detector 18 or 24. The X-ray image acquisition computer 28 transfers 2-D images of the anatomical region to a registration computer system 30. In certain embodiments, where devices (e.g., catheters) are inserted within the body of the subject 20, the 2-D X-ray image may include an image of the device (e.g., catheter) and a direction of the catheter relative to the 2-D image.

The system 10 also includes a catheter monitoring/control system 32, a respiratory monitoring system 34, and a heart monitoring system 36. The catheter monitoring/control system 32 controls and is coupled to a catheter 38 disposed within the body of the subject 20. In particular, the catheter 38 may be advanced through the vessel system of the subject 20 into the heart. The catheter 38 includes a plurality of transducers and/or sensors 40 disposed on an operative end or tip 42 of the catheter 38 for collecting data or information. The sensors 40 may include a contact/orientation sensor 44 to provide a measurement of contact between the catheter 38 and a surface of a tissue (e.g., vessel or wall) within the subject 20. For example, the sensor 44 may measure force and/or impedance between the catheter 38 and a surface of a tissue. In addition, the contact/orientation sensor 44 provides a direction of the catheter 38 with respect to the surface of the tissue contacted. The information collected from the contact/orientation sensor 44 may be used in conjunction with the direction of the catheter 38 from the image of the catheter 38 in the generated X-ray image to derive a position and orientation of the catheter 38. Another sensor 40 includes an electrophysiological sensor 46. The electrophysiological sensor 46 may provide an electrical voltage of the tissue (e.g., heart) or electrical waveforms (e.g., electrocardiogram (ECG)). An additional sensor 40 includes an elasticity sensor 48 to determine the elasticity of a wall (e.g., vessel). The sensor 40 may include other sensors 50 as well. For example, the other sensors 50 may generate an image with ultrasound or another type of bandwidth (e.g., UV, visible, and infrared light spectrums). Other sensors 50 may also provide a time of measurement by the catheter 38. The sensors 40 may be provide the collected data or information to the catheter monitoring/control system 32. Alternatively, the sensors 40 may provide the collected data or information to other systems (e.g., heart monitoring system 36). The catheter monitoring/control system transfers the collected data or information to the registration computer system 30.

The respiratory monitoring system 34 is also coupled to the subject 20. The respiratory monitoring system 34 utilizes the 2-D images generated by the X-ray image acquisition computer 28 to determine a phase of respiratory cycle. The registration computer system 30 may utilize the data representing the phase of the respiratory cycle from the respiratory monitoring system 34 to instruct the X-ray image acquisition computer 28 to generate 2-D images during a predetermined phase of the respiratory cycle (i.e., respiratory gating).

Further, the heart monitoring system 36 is coupled to the subject 20. The heart monitoring system 36 generates an ECG signal indicative of a cardiac cycle of the subject 20 and provides the signal to the registration computer system 30. In response to the ECG signal the registration computer system 30 may instruct the X-ray image acquisition computer 28 to generate 2-D images when the heart has a predetermined phase of a cardiac cycle (i.e., cardiac gating). Alternatively, the registration computer system 30 may utilize intracardiac electrograms obtained from the electrophysiological sensor 46 of the catheter 38 to instruct the X-ray image acquisition computer 28 to generate 2-D images when the heart has a predetermined phase of a cardiac cycle. Methods and techniques for generating an image relative to the cardiac cycle (i.e., cardiac gating) and the respiratory cycle (i.e., respiratory gating) may be found in U.S. Application Pub. No. 2009/0208079 entitled “METHOD FOR GENERATING A REGISTERED IMAGE RELATIVE TO A CARDIAC CYCLE AND A RESPIRATORY CYCLE OF A PERSON” filed on Oct. 30, 2007, which is hereby incorporated into the present disclosure by reference in its entirety.

The data collected from the respiratory monitoring system 34 and the heart monitoring system 36 may be used to reduce inaccuracies due to motion of the subject's anatomy by synchronizing the generated X-ray images employed in the present disclosure. Methods and techniques for reducing inaccuracies due to motion of the subject's anatomy via synchronization may be found in U.S. Application Pub. No. 2011/0019878 entitled “SYSTEM AND METHOD TO COMPENSATE FOR RESPIRATORY MOTION IN ACQUIRED RADIOGRAPHY IMAGES” filed Jul. 23, 2009, and U.S. Application Publ. No. 2008/0240536 entitled “METHOD OF DETECTION AND COMPENSATION FOR RESPIRATORY MOTION IN RADIOGRAPHY CARDIAC IMAGES SYNCHRONIZED WITH AN ELECTROCARDIOGRAM SIGNAL” filed Mar. 25, 2008, both of which are incorporated into the present disclosure by reference in their entirety.

The X-ray image acquisition computer 28, the catheter monitoring/control system 32, the respiratory monitoring system 34, and the heart monitoring system 36 are all coupled to the registration computer system 30. As described in greater detail below, the registration computer system 30 receives the data from these multiple sources to generate a single dataset.

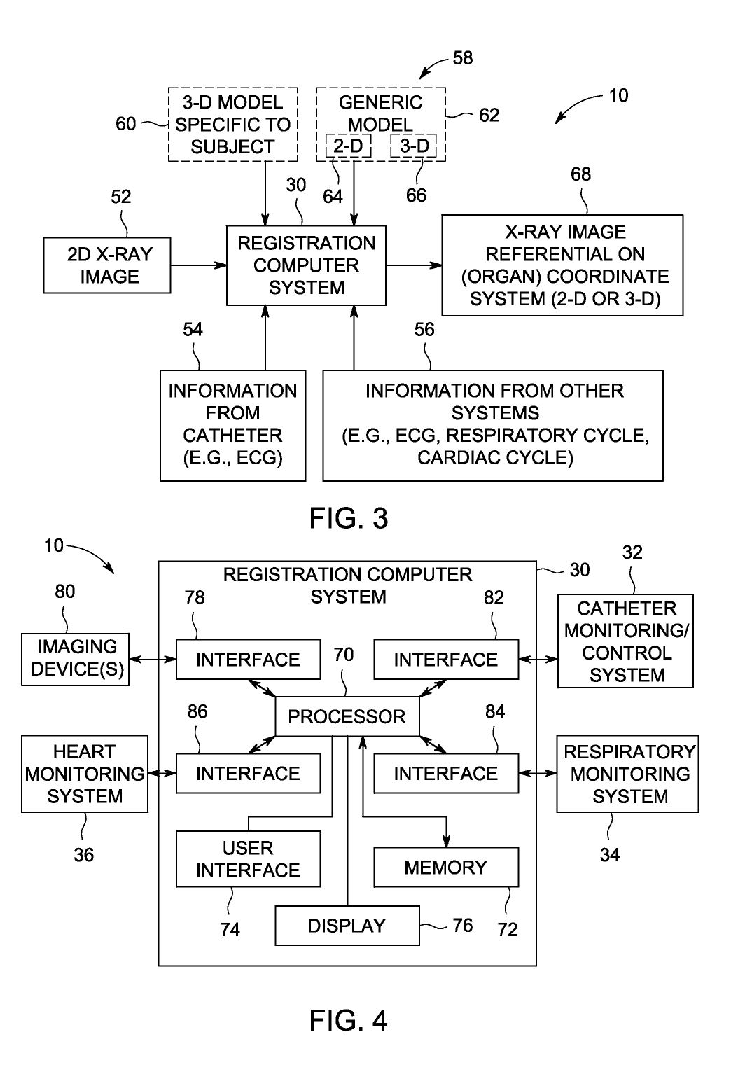

FIG. 3 illustrates the collection of data from multiple sources and the integration of the data into a single dataset by the registration computer system 30 of system 10. As illustrated, the registration computer system 30 receives a 2-D X-ray image 52, for example, generated by the X-ray imaging system 12 via the X-ray image acquisition computer 28. In certain embodiments, the registration computer system 30 may receive multiple X-ray images 52, for example, generated via bi-plane imaging. As mentioned above, the 2-D X-ray image 52 includes an image of the catheter 38 and a direction of the catheter 38. The registration computer system 30 also receives data or information 54 collected from the sensors 40 of the catheter 38 via the catheter monitoring/control system 32. The collected information 54 from the sensors 40 may include a measurement of a contact (e.g., force and/or impedance measurement) between the catheter 38 and a surface of a tissue (e.g., vessel or wall) within the subject 20, a direction of the catheter 38 with respect to the surface of the tissue contacted, an electrical voltage of the tissue (e.g., heart) or electrical waveforms (e.g., ECG), an elasticity of a wall (e.g., vessel), images (e.g., ultrasound), a time of measurement by the catheter 38. In addition, the registration computer 30 also receives data or information 56 collected from other systems such as the respiratory monitoring system 34 and the heart monitoring system 36. The collected information 56 from the other systems may include a respiratory cycle, a cardiac cycle, or an electrocardiogram of the subject 20.

The registration computer system 30 receives a model of an anatomy 58. The model of the anatomy 58 may be received from the registration computer system 30 via another system component (e.g., X-ray image acquisition computer 28), or via an internal or external network that includes a picture archiving and communications system (PACS), radiology department information system (RIS), and/or hospital information system (HIS). Alternatively, the model of the anatomy 58 may be stored within a memory of the registration computer system 30. The model of the anatomy 58 may include a 3-D model 60 specific to the subject 20. For example, the 3-D model 60 may have been generated by another imaging device or system separate from imaging system 12. The other imaging system may include a 3-D imaging system such a computed tomography (CT) system, a magnetic resonance imaging (MRI) system, or ultrasound imaging system. In the absence of a 3-D model 60 specific to the subject 20, a generic model 62 of the anatomy of interest may be used. The generic model 62 may be a 2-D generic model 64 or a 3-D generic model 66. The 2-D generic model 64 corresponds to a plane of the anatomy along the projection angle used for the generated X-ray image 52. The 3-D generic model 66 need not correspond to the projection angle used for the generated X-ray image 52.

The registration computer system 30 is configured to generate an X-ray image information model or X-ray image referential 68 on a coordinate system (e.g., organ coordinate system) based on the 2-D X-ray image 52, the collected information 54 and 56, and the model of the anatomy 58. In particular, the registration computer system 30 merges the collected information 54 and 56 to the 2-D X-ray image 52 to generate the X-ray image referential 68. The collected information 54 and 56 may be added to the X-ray image referential 68 during or after an interventional procedure. In addition, subsequent or follow-up images may be merged into the X-ray image referential 68 subsequent to the generation of the X-ray image referential 68. For example, MR images may be merged into the X-ray image referential 68 to correlate information generated during the interventional procedure (e.g., burn case information) with information generated from the MR images (e.g., edema analysis) to compare the quality of tissue transformation.

The X-ray image referential 68 may include a 2-D X-ray image referential if the generic 2-D model 64 is used to generate the referential 68. Alternatively, the X-ray image referential 68 may include a 3-D X-ray image referential if the 3-D model 60 specific to the subject 20 or the generic 3-D model 66 is used to generate the referential 68. In some embodiments, the coordinate system utilized in the X-ray image referential 68 includes a coordinate system associated with the 2-D array of image pixels from the entire image 52. Alternatively, the registration computer system 30 may translate the coordinates associated with the 2-D array of image pixels to an organ coordinate system, for example, specific to the organ (e.g., heart) in the image 52. Further, the registration computer system 30 may translate the coordinates associated with the organ coordinate system back to the coordinates associated with the 2-D array of image pixels. The 2-D X-ray image referential provides at least X- and Y-coordinates. In some embodiments, the 2-D X-ray image referential may provide a Z-coordinate due to the registration computer system 30 transforming (e.g., via homography) the 2-D array of image pixels into a 3-D reconstructed coordinate system. The 3-D X-ray image referential provides X-, Y-, and Z-coordinates.

The X-ray referential image 68 collects the data from the multiple sources into a single dataset. As described in greater detail below, the X-ray image referential 68 serves as a single reference or access point for the generated X-ray image 52 and any collected information related to the image 52. The X-ray image referential 68 may display a trace of the catheter 38 through the anatomy (e.g., heart) of the subject 20. In addition, the X-ray image referential 68 may display the current position and orientation of the catheter 38 as well as past positions and orientations of the catheter with respect to other points along the trace. In some embodiments that utilize a 2-D X-ray image referential, the referential may be transformed via homography by the registration computer system 30 to project the position and orientation of the catheter 38 in a different plane from a plane of the X-ray image 52.

Further, the X-ray image referential 68 allows a user to select points along the trace to obtain the collected information 54 and 56 associated with the trace. For example, the X-ray image referential 68 may include an X-ray image, 3D and/or 4D information related to the image, position and orientation of the catheter, electrophysiological data at certain points along the trace, parameters related to the catheter at certain points along the trace, a respiratory cycle, a cardiac cycle, images (e.g., ultrasound), and other information all within the X-ray image referential 68. The X-ray image referential 68 may also include a hierarchical structure that allows immediate visualization or direct access to the collected information (e.g., via menus, tables, links, etc.) associated with each selected point along the trace of the catheter. In addition, the X-ray image referential 68 may allow editing of the information within the referential 68.

FIG. 4 illustrates the structure of the registration computer system 30 of system 10 in greater detail. The registration computer system 30 includes a processor 70, a memory 72, a user interface 74, and a display 76. The processor 70 includes an interface 78 (e.g., interface circuitry) to receive image data from one or more imaging devices 80. For example, the processor 70 may receive the 2-D X-ray image 52 of the subject 20 from the fluoroscopic imaging system 12 (e.g., via X-ray image acquisition computer 28) and/or a 3-D X-ray model 60 of the subject from another imaging system. The processor 70 also includes an interface 82 (e.g., interface circuitry) to receive the collected information 54 from the catheter monitoring/control system 32. In addition, the processor 70 includes interfaces 84 and 86 (e.g., interface circuitry) to receive the collected information 56 from the respiratory monitoring system 34 and the heart monitoring system 36.

The processor 70 is coupled to the memory 72. The processor 70 can be arranged independent of or integrated with the memory 72. The processor 70 is generally operable to execute program instructions representative of acts or steps described herein and stored in the memory 72. The processor 70 may include a single central processor or multiple processors. The processor 70 is configured to receive input data or information or communicate output data. More specifically, the processor 70 is configured to generate the X-ray image referential 68 on a coordinate system (e.g., organ coordinate system) by merging the model of an anatomy 58 and the collected information 54 and 56 to the 2-D X-ray image 52. In some embodiments, the processor 70 is configured to translate the coordinates associated with the 2-D array of image pixels for the entire image 52 to an organ coordinate system, for example, specific to the organ (e.g., heart) in the image 52. In certain embodiments, the processor 70 is configured to direct acquisition of the 2-D X-ray image 52 during a particular phase of a respiratory cycle and/or a cardiac cycle as described above. In some embodiments, the processor 70 is also configured to reducing inaccuracies due to motion of the subject's anatomy via synchronization as described above. In addition, the processor 70 is configured to receive a selection of a point along the trace of the catheter 38 in the X-ray image referential 68. Further, the processor 70 is configured to provide the current position and orientation of the catheter 38 and collected information 54 and 56 on the X-ray image referential 68 for the current point along the trace. Also, the processor 70 is configured to provide the position and orientation of the catheter 38 and collected information 54 and 56 on the X-ray image referential 68 for any selected point along the trace. In certain embodiments, the processor 70 is configured to transform the X-ray image referential 68 via homography to project the position and orientation of the catheter 38 in a different plane from a plane of the 2-D X-ray image 52.

The memory 72 may comprise one or more tangible, non-transitory computer readable media operable to store multiple computer-readable program instructions for execution by the processor 70. By way of example, such media may comprise RAM, ROM, PROM, EPROM, EEPROM, Flash, CD-ROM, DVD, or other known computer-readable media or combinations thereof which can be used to carry or store desired program code in the form of instructions or data structures and which can be accessed by a general purpose or special computer or other machine with a processor. The memory 72 is also configured to store data generated or received by the registration computer system 30. For example, the memory 72 may store the model of the anatomy 58, the 2-D X-ray image 52, and collected information 54 and 56.

As mentioned above, the registration computer system 30 includes the user interface 74 and the display 76. The user interface 74 may include a keyboard and/or mouse, as well as other devices such as printers or other peripherals for reproducing hardcopies of the information from the X-ray image referential 68. The user interface 74 enables the user to input directives, to navigate the X-ray image referential 68, and to edit the referential 68. For example, the user may select points along the trace of the catheter 38, select information for display from pull-down menus or tables, and rotate the model of the organ (e.g., heart) within the X-ray image referential 68. The display 76 is configured to display the X-ray image referential 68 and any associated information selected from the X-ray image referential 68. For example, the display 76 may display the current position and orientation of the catheter 38, selected points along the trace of the catheter 38, and collected information 54 and 56 as well as positions and orientations of the catheter 38 associated with the selected points along the trace.

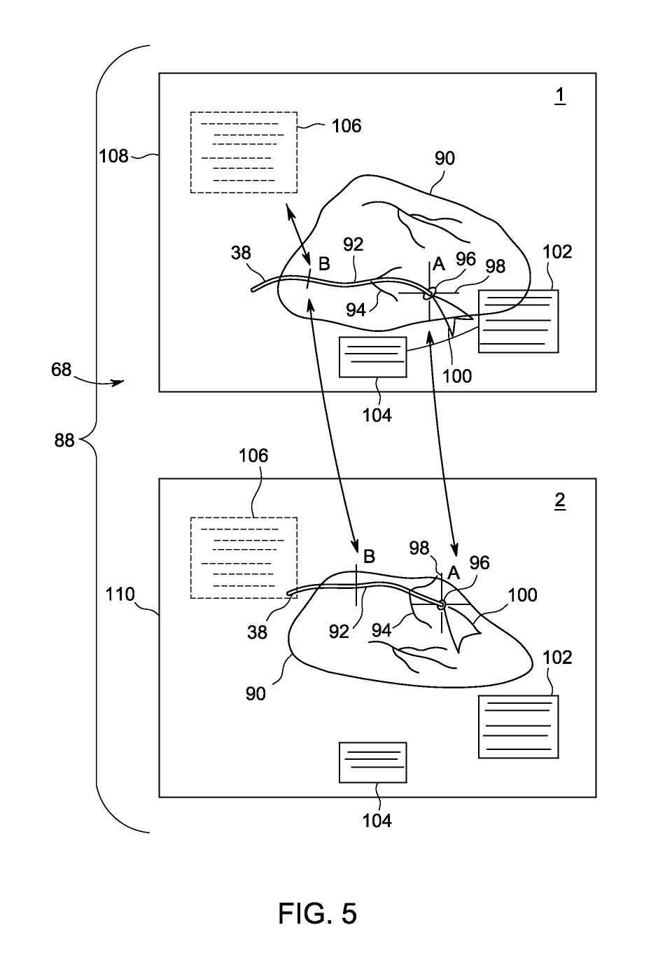

FIG. 5 illustrates an example of a screen 88 of the X-ray image referential 68 that may be displayed on the display 76. The X-ray image referential 68 includes a model organ 90 (e.g., heart) generated by the merging of the 2-D X-ray image 52 and the model of the anatomy 58. The X-ray image referential 68 also displays a trace 92 of the catheter 38 through a vessel system 94 of the model organ 90. The current location of the catheter 38 may be illustrated by a dot 96 and/or cross-hair 98. In FIG. 5, point A illustrates the current location of the catheter 38. The X-ray image referential 68 provides a hierarchical structure for accessing information related to the interventional procedure for the heart. For example, using pointer 100 via the user interface 74 to select point A along the trace 92, a pop-up screen of information 102 appears on the X-ray image referential 68 displaying the position and orientation of the catheter 38 as well as collected information 54 and 56 (e.g., a first tier of information) associated with point A. For example, the collected information 54 and 56 may include an X-ray image, electrophysiological data, parameters related to the catheter, a respiratory cycle, a cardiac cycle, images, time of measurement, impedance, and other information. The pop-up screen 102 may include an image, hyperlinks, a pull-down menu, a table, and/or a list. From the pop-up screen 102, additional information (e.g., second tier of information) may be selected for display in a separate pop-up screen 104. Besides information from point A, other points may be selected along the trace 92. For example, point B may be selected and a pop-up screen 106 similar to screen 102 appears on the X-ray image referential 68 displaying the position and orientation of the catheter 38 at point B along with the collected information 54 and 56 associated with point B. Additional information may be selected from pop-up screen 106 for display. In certain embodiments, the X-ray image referential 68 enables the user to edit the information within the referential 68.

As mentioned above, the X-ray image referential 68 may include a 2-D or a 3-D X-ray image referential depending on the model of the anatomy 58 used to generate the referential 68. As illustrated, the X-ray image referential 68 includes one or more smaller screens. For example, a 2-D X-ray image referential may include a single screen 108 (e.g., screen 1). In a 3-D X-ray image referential, the heart model 90 may be rotated. To illustrate the rotation of the heart model, the 3-D X-ray image referential may include an additional screen 110 (e.g., screen 2) that includes a mirror image of the heart model 90 as it is rotated. The user may interchangeably interact with screens 108 and 110.

The X-ray image referential 68 may be a useful tool in many applications. For example, the X-ray image referential 68 may used to monitor physiological (e.g., electrical) leakage along a particular trace of a catheter through the heart. For example, during an interventional procedure, an ablation catheter may be used to generate a first ablation burn (e.g., for calibration) at a site of interest along the trace. Measurements such as time, energy, impedance, force, and/or other measurements may be collected at the first ablation burn site. In addition, the physical size of the burn may be determined (e.g., using 2-D intracardiac echocardiography imaging). The measured size of the first ablation burn and/or the other collected information may be used as a standard for comparison to subsequent burn sites along the trace. Subsequent burn sites that do not conform to the parameters of the first ablation burn site may be marked in a different color dot or by some other method to differentiate the non-conforming burn site along the trace on the X-ray referential 68. Further, the distance between adjacent ablation points or burn sites (e.g., distance between the centers of the burn sites) may be measured. If the distance exceeds the institutional quality standard for spacing or distance the ablation point is marked along the trace in the X-ray image referential 68. Ablation points may also be marked as areas that meet the desired specification but that need to be subsequently monitored in the event of a subsequent physiological condition (e.g., arrhythmia). All of the collected information related to the ablation points may be added to the X-ray image referential 68 and displayed and/or accessed via the referential 68. A subsequent mapping catheter may be used to monitor all of the ablation points along the trace (e.g., for electrical leakage) to determine the need for a further ablation point to terminate leakage current. For example, the information collected using the mapping catheter may be compared to the information collected with the ablation catheter.

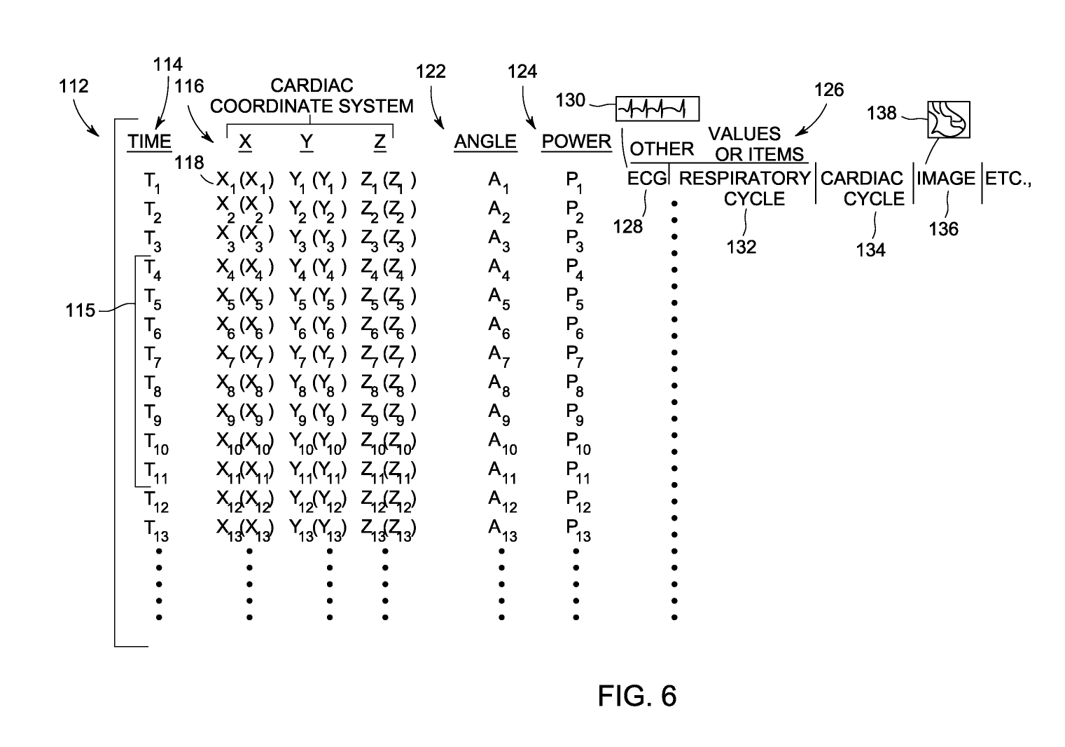

An example of information in screens 102, 104, or 106 of the X-ray image referential 68 is illustrated in a table 112 in FIG. 6. As mentioned above, the display of the information on the screens 102, 104, and 106 may vary. For example, the information may be displayed in a menu or list with additional pull-down menus or hyperlinks to other information. In addition, the information displayed in each screen 102, 104, and 106 may vary. The table 112 includes a time of measurement 114 (e.g., T). Certain times of measurement 114 may be identified in the table 112 as indicated by reference numeral 115 (e.g., via bracketing, highlighting, etc.) to indicate these measurements 114 fall within the sweet spot in the cardiac cycle (i.e., when the myocardial strain effects cancel out). In addition, the table 122 includes a coordinate system 116 (e.g., cardiac coordinate system) for the location of the catheter 38 including at least X- and Y-coordinates corresponding to the time of measurement 114. In a certain embodiments, the coordinate system 116 also includes a Z-coordinate. The coordinate system 116 may include raw values 118 (e.g., X, Y, and Z) and/or corrected values 120 (e.g., (X), (Y,), and (Z)). The corrected values 120 may reflect corrections for movement of the subject 20 as described above. The table 112 further includes an orientation or angle 122 (e.g., A) for the catheter 38 as well as a power measurement 124 (e.g., P) for the catheter 38 corresponding to the time of measurement 114. The table 112 may further include other values or items 126 (e.g., force or impedance measurement for catheter contact). For example, a link 128 to an ECG recording 130 may be listed. Selecting the link 128 displays the ECG recording 130 on the X-ray image referential 68. The ECG recording 130 may include the period before during and after the corresponding time of measurement 114. The table 112 may also specify the respiratory cycle 132 and/or cardiac cycle 134 corresponding to the time of measurement 114. In addition, the table 112 may include a link 136 to an image 138, for example, of the heart. The image 138 may include an X-ray image, ultrasound image, or image generated with another type of bandwidth (e.g., UV, visible, and infrared light spectrums). Additional items 126 included in the table 112 may include an electrical voltage of a tissue (e.g., heart), elasticity of a wall of the tissue, or the energy applied during ablation by the catheter 38. The above items in the table 112 are only examples and are not intended to be an exhaustive list of items. Other items not shown may be included in the table 112.

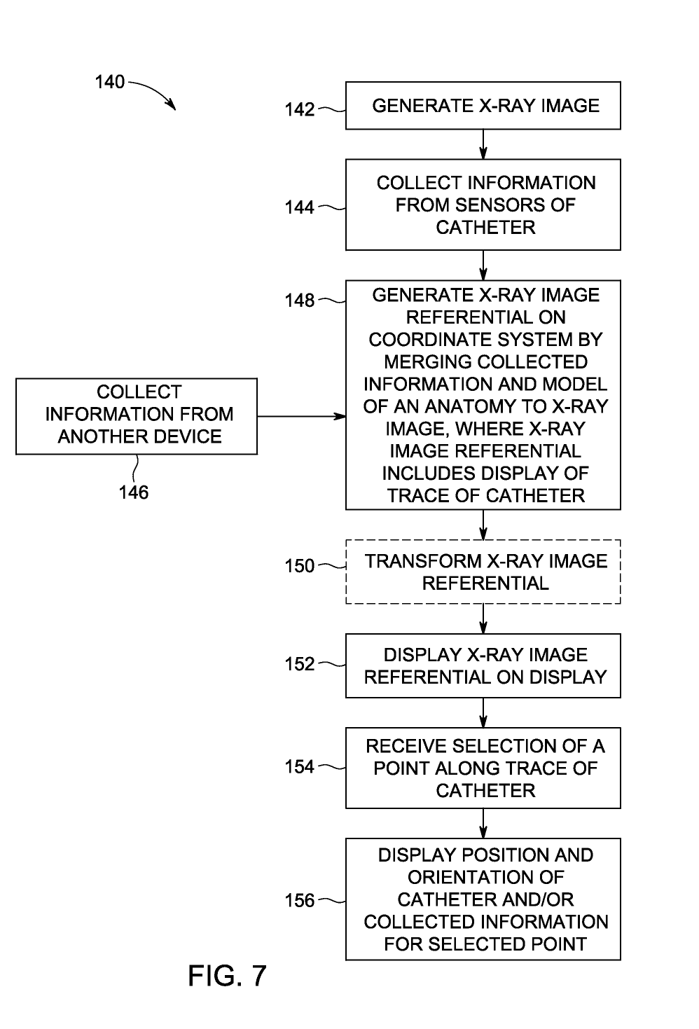

FIG. 7 illustrates a flow diagram of an exemplary method 140 for integrating multiple data sources into the X-ray image referential 68. The method 140 includes generating the X-ray image 52 of the subject 20 (block 142), such as a 2-D X-ray image, using imaging system 12 (e.g., fluoroscopy imaging system). The method 140 further includes collecting information 54 (e.g., electrophysiological measurements, contact, etc.) as described above from sensors 40 of the catheter 38 disposed within the subject 20 (e.g., disposed within the heart) (block 144). In certain embodiments, the method 140 includes collecting information 56 (e.g., cardiac cycle, respiratory cycle, etc.) as described above from other devices or systems (e.g., heart respiratory monitoring system 34 and/or heart monitoring system 36) (block 146). In addition, the method 148 includes generating the X-ray image referential 68 on a coordinate system (e.g., organ coordinate system) by merging collected information 54 and/or 56 and the model of the anatomy 58 to the X-ray image 52 (block 148). The X-ray image referential 68 displays the trace 92 of the catheter 38. The registration computer system 30 may receive the image 52, collected information 54 and 56, and model of the anatomy 58 and generate X-ray referential 68. As mentioned above, the model of the anatomy 58 may include the 3-D model 60 specific to the subject 20 generated by another imaging device, generic 2-D model 64, or generic 3-D model 66. The X-ray image referential 68 may be 2-D or 3-D depending on the type of model of the anatomy 58 used. If the X-ray image referential 68 is a 2-D X-ray image referential, the method 140 may include transforming the X-ray referential 68 (e.g., the model 90 within the referential) via homography to project the position and orientation of the catheter 38 in a different plane from a plane of the X-ray image 52.

Upon generation of the X-ray image referential 68 (block 148), the method 140 includes displaying the X-ray image referential 68 (block 152) on the display 76 of the registration computer system 30. The method 140 also includes receiving a selection of a point along the trace 92 of the catheter 38 (block 154). Upon selection of the point (block 154), the method 140 includes displaying the position and orientation of the catheter 38 and/or collected information 54 and 56 for the selected point on the X-ray image referential 68 (block 156). In certain embodiments, the position and orientation of the catheter 38 may be displayed for the current point along the trace 92 of the catheter 38 without selection of the current point.

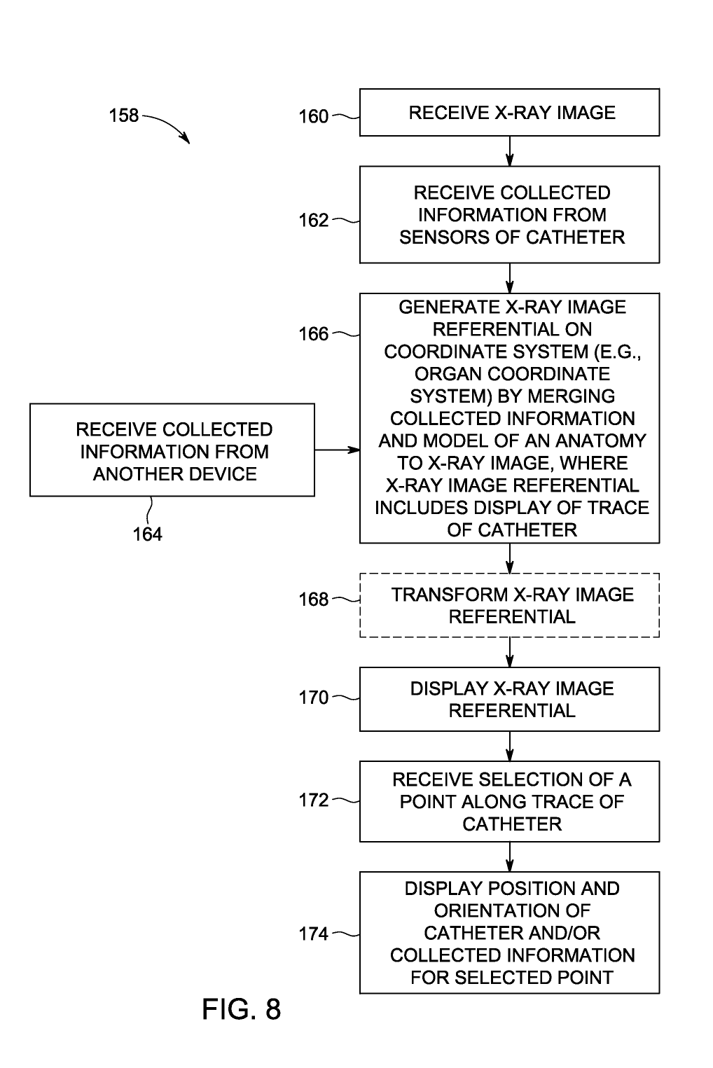

Similarly, FIG. 8 illustrates a process diagram of an exemplary program 158 for integrating multiple data sources into an X-ray image referential 68. As mentioned above, the program 158 (e.g., code) may be stored on a tangible, non transitory computer medium of the memory 72 of the registration computer system 30. One or more processors 70 of the registration computer system 30 may implement the steps or acts of the program 158. The program 158 includes receiving the X-ray image 52 of the subject 20 (block 160), such as a 2-D X-ray image, from the imaging system 12 (e.g., fluoroscopy imaging system) via the X-ray image acquisition computer 28. The program 158 further includes receiving collected information 54 (e.g., electrophysiological measurements, contact, etc.) as described above from sensors 40 of the catheter 38 disposed within the subject 20 (e.g., disposed within the heart) (block 162) via the catheter monitoring/control system 32. In certain embodiments, the program 158 includes receiving collected information 56 (e.g., cardiac cycle, respiratory cycle, etc.) as described above from other devices or systems (e.g., heart respiratory monitoring system 34 and/or heart monitoring system 36) (block 168). In addition, the program 158 includes generating the X-ray image referential 68 on a coordinate system (e.g., organ coordinate system) by merging collected information 54 and/or 56 and the model of the anatomy 58 to the X-ray image 52 (block 166). The X-ray image referential 68 displays the trace 92 of the catheter 38. As mentioned above, the model of the anatomy 58 may include the 3-D model 60 specific to the subject 20 generated by another imaging device, generic 2-D model 64, or generic 3-D model 66. The X-ray image referential 68 may be 2-D or 3-D depending on the type of model of the anatomy 58 used. If the X-ray image referential 68 is a 2-D X-ray image referential, the program 158 may include transforming the X-ray referential 68 (e.g., the model 90 within the referential) via homography to project the position and orientation of the catheter 38 in a different plane from a plane of the X-ray image 52.

Upon generation of the X-ray image referential 68 (block 166), the method 140 includes displaying the X-ray image referential 68 (block 170) on the display 76 of the registration computer system 30. The program 158 also includes receiving a selection of a point along the trace 92 of the catheter 38 (block 172). Upon selection of the point (block 172), the method 140 includes displaying the position and orientation of the catheter 38 and/or collected information 54 and 56 for the selected point on the X-ray image referential 68 (block 174). In certain embodiments, the position and orientation of the catheter 38 may be displayed for the current point along the trace 92 of the catheter 38 without selection of the current point.

Technical effects of the disclosed embodiments include providing methods and systems to collect and integrate data from multiple sources into a single dataset. Specifically, the single dataset includes the X-ray image information model or X-ray image referential 68 generated from the 2-D X-ray image 52 (e.g., heart of subject 20), the model of the anatomy 58 (e.g., 2-D or 3-D model) corresponding to the region of interest in the image 52, and collected information 54 and 56 from multiple sources (e.g., sensors 40 of the catheter 38, respiratory monitoring system 34, and heart monitoring system 36). The X-ray image referential 68 displays the trace 92 of the catheter 38. The collected information 54 and 56 and the position and orientation of catheter 38 may be displayed for the current point and selected points along the trace 92 of the catheter. The X-ray image referential 68 provides a single visual reference or access point to all of the data associated with an interventional procedure. Further, the X-ray image referential 68 provides a cost effective and user-friendly navigable source that eliminates the need to switch between and search multiple sources for the data associated with the interventional procedure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for integrating multiple data sources into an X-ray image referential, comprising:
generating an X-ray image of a subject;
collecting information from sensors of a catheter disposed within the subject;
generating the X-ray image referential on a coordinate system by merging a model of an anatomy and the collected information from the sensors of the catheter to the X-ray image, wherein the X-ray image referential includes a display of a trace of the catheter; and
displaying the X-ray image referential on a display.

2. The method of claim 1, wherein the X-ray image comprises a 2-D image having an image of the catheter and a direction of the catheter relative to the 2-D image.

3. The method of claim 1, wherein the collected information comprises a measurement of contact between the catheter and a surface of a tissue within the subject, an electrical voltage of the tissue, an electrocardiogram, an image acquired by the catheter, or a time of measurement by the catheter.

4. The method of claim 1, comprising collecting additional information from another device, wherein the additional collected information comprises an electocardiogram, a cardiac cycle, or a respiratory cycle of the subject.

5. The method of claim 1, wherein the coordinate system comprises an organ coordinate system.

6. The method of claim 1, wherein the model of the anatomy comprises a 3-D model generated of the subject by an imaging device.

7. The method of claim 1, wherein the model of the anatomy comprises a generic 3-D model.

8. The method of claim 1, wherein the model of the anatomy comprises a generic 2-D model, and the method comprises transforming the X-ray image referential via homography to project a position and orientation of the catheter in a different plane from a plane of the X-ray image.

9. The method of claim 1, comprising displaying a position and orientation of the catheter on the X-ray image referential.

10. The method of claim 1, comprising receiving a selection of a point along the trace of the catheter.

11. The method of claim 10, comprising displaying on the X-ray image referential the collected information for the selected point along the trace.

12. The method of claim 10, comprising displaying a position and orientation of the catheter for the selected point along the trace.

13. The method of claim 1, wherein the X-ray image referential comprises a 2-D X-ray image referential.

14. The method of claim 1, wherein the X-ray image referential comprises a 3-D X-ray image referential.

15. A system, comprising:
an imaging device configured to generate an X-ray image of a subject;
a catheter disposed within the subject, wherein the catheter includes sensors configured to collect information;
a processor configured to generate an X-ray image referential on an organ coordinate system by merging a model of an anatomy and collected information from the sensors of the catheter to the X-ray image, wherein the X-ray image referential includes a display of a trace of the catheter; and
a display configured to display the X-ray image referential.

16. The system of claim 15, wherein the collected information comprises a measurement of contact between the catheter and a surface of a tissue within the subject, an electrical voltage of the tissue, an electrocardiogram, an image acquired by the catheter, or a time of measurement by the catheter.

17. The system of claim 15, wherein the model of the anatomy comprises a 3-D model generated of the subject by an imaging device.

18. The system of claim 15, wherein the model of the anatomy comprises a generic 2-D or 3-D model.

19. The system of claim 15, wherein the display is configured to display a position and orientation of the catheter on the X-ray image referential.

20. The system of claim 15, wherein the processor is configured to receive a selection of a point along the trace of the catheter, and the display is configured to display the selected point along the trace, the collected information for the selected point, or a position and orientation of the catheter for the selected point along the trace on the X-ray image referential.

21. The system of claim 15, wherein the X-ray image referential comprises a 2-D or 3-D X-ray image referential.

22. The system of claim 15, wherein the model of the anatomy comprises a generic 2-D model, and the processor is configured to transform the X-ray image referential via homography to project a position and orientation of the catheter in a different plane from a plane of the X-ray image.

23. A computer program, stored on a tangible, non-transitory computer readable medium, for integrating multiple data sources into an X-ray image referential, the program constructed and arranged to:
receive an X-ray image of a subject and collected information from sensors of a catheter disposed within the subject;
generate the X-ray image referential on an organ coordinate system by merging a model of an anatomy and the collected information from the sensors of the catheter to the X-ray image, wherein the X-ray image referential includes a display of a trace of the catheter; and
display the X-ray image referential.

24. The computer program of claim 23, wherein the program is constructed and arranged to display a position and orientation of the catheter on the X-ray image referential.

25. The computer program of claim 23, wherein the program is constructed and arranged to display the collected information for a selected point along the trace on the X-ray image referential.
